(19) United States
(12) Patent Application Publication
(10) Pub. No.: US 2022/0074893 A1
(43) Pub. Date: Mar. 10, 2022

(54) DETECTING STRESS-STRAIN IN METAL COMPONENTS

(71) Applicant: PICA: Pipeline Inspection and Condition Analysis Corporation, Edmonton (CA)

(72) Inventors: David E. Russell, Sherwood Park (CA); Yuwu Yu, Edmonton (CA); Ad Shatat, Edmonton (CA); Martin Korz, Edmonton (CA)

(73) Assignee: PICA: Pipeline Inspection and Condition Analysis Corporation, Edmonton (CA)

(21) Appl. No.: 17/447,277

(22) Filed: Sep. 9, 2021

Related U.S. Application Data
(60) Provisional application No. 63/076,606, filed on Sep. 10, 2020.

(51) Int. Cl. G01N 27/90; G01N 33/204

(52) U.S. Cl. CPC G01N 27/90; G01N 33/204

(57) ABSTRACT

A system for detecting and quantifying changes in the stress-strain state of a ferrous structure includes an exciter coil system is positioned to generate an AC magnetic field that couples into the ferrous structure. A detector apparatus is positioned relative to the excited to detect an eddy current magnetic field resulting from the AC magnetic field generated by the exciter coil system. An analyzer compares the eddy current magnetic field parameters detected by the detector apparatus with the direct AC magnetic field transmitted by the exciter coil system and correlates changes in the parameters of the eddy current magnetic field with the stress-strain on the ferrous structure.

### CROSS-REFERENCE TO RELATED APPLICATION

This application claims the benefit of U.S. Provisional Application No. 63/076,606, filed Sep. 10, 2020, the entire contents of which are expressly incorporated herein by reference.

### FIELD OF THE INVENTION

This invention relates to methods for non-destructive testing of steel components and especially pipe and pipelines. More particularly, this invention relates to the use of remote field eddy current (RFEC, also known as Remote Field Testing, RFT), or Near Field Eddy Current Testing (NFEC) or (NFT), to detect local changes in the stress-strain state of steel pipes and pipelines and other ferrous objects where an external or internal load has changed. The invention will work through coatings, fire-proofing, cement layers, and insulation at distances of up to 4″ from the ferrous metal.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

This invention discloses systems, apparatus, and methods for detecting local anomalies in the stress-strain state of metal pipeline or other ferrous structures. The apparatus includes alternating current flux production means and detector means disposed proximal to the surface of a steel or other ferrous based cylinder or pipe or structure. The apparatus is traversed axially through the inside of the pipe or along the outside of a pipeline that is manufactured of steel pipes or of cylinders joined together by welds or other joint types such as flanges, bell and spigot connections, riveted over-lapped joints etc.

The apparatus contains an exciter means which generates an alternating current signal which couples into the pipe and any reinforcing or pre-stress wires or bars, and creates electrical eddy currents which radiate away from the exciter means, within and through the pipe wall thickness and any reinforcing wires or bars.

At a distance from the exciter means, which can vary from two inches to up to five pipe diameters, at least one detector means receives the induced signal. The received signal is compared to the exciter means. In this regard, two parameters are measured and recorded: signal travel time (phase lag) and signal amplitude, both of which are indirect measurements of four characteristics of the steel/ferrous pipe wall and reinforcing wires or bars. These characteristics are:
Wall thickness
Electrical Conductivity
Relative Magnetic Permeability
Transformer Coupling

It is the measurement of the relative magnetic permeability and relating it to the change in the stress strain state of the material that forms the basis of the present system and method.

Relative magnetic permeability is a characteristic of steels/ferrous materials that is an indirect measurement of the local stress-strain state of the metal.

If the local stress-strain state of a steel/ferrous pipeline is affected by an external load or the local absence of a constant load, the relative magnetic permeability of the steel/ferrous structure is changed and is measurable by suitable exciter-detector means.

In accordance with one embodiment of the present disclosure, a system is provided for detecting and quantifying changes in the stress-strain state of a ferrous structure. The system includes an exciter to generate an AC magnetic field that couples into the ferrous structure, a detector apparatus to detect an eddy current magnetic field resulting from the AC magnetic field generated by the exciter coil system, and an analyzer that compares the eddy current magnetic field parameters detected by the detector apparatus with the direct AC magnetic field transmitted by the exciter coil system and correlates changes in the parameters of the eddy current magnetic field with the stress-strain on the ferrous structure.

In accordance with one embodiment of the present disclosure, a system is provided for detecting and quantifying the condition of a structure that is at least partially composed of ferrous substrate material or ferrous wires for pre-stressing the structure. The system includes an exciter coil system energized with an alternating current signal to generate an alternating magnetic field that couples into the ferrous structure or ferrous wires, a detector apparatus to detect an eddy current magnetic field resulting from the alternating magnetic field generated by the exciter coil system, and an analyzer that compares the eddy current magnetic field parameters detected by the detector apparatus with the alternating magnetic field transmitted by the exciter coil system and correlates changes in the parameters of the eddy current magnetic field with: (a) changes in the wall thickness of the ferrous structure or breaks in the wire, as well as (b) changes in the stress-strain on the ferrous structure or the structure that is pre-stressed by the wires.

In any of the embodiments described herein, wherein the magnetic field parameters that are analyzed include the amplitude and phase lag of the voltage of the eddy current magnetic field detected by the detector apparatus.

In any of the embodiments described herein, wherein the analyzer determines changes in the magnetic permeability of the ferrous structure based on the amplitude and phase lag of the voltage of the eddy current magnetic field detected by the detector apparatus, and correlates the changes in magnetic permeability with the level of stress-strain on the ferrous structure.

In any of the embodiments described herein, wherein the frequency of the generated AC magnetic field is in the range of 0.5 to 1000 hertz.

In any of the embodiments described herein, wherein the ferrous structure is selected from a group including: ferrous pipe, ferrous tubing, ferrous tanks, ferrous pressure vessels, prestressed concrete cylinder pipe, ferrous beams; ferrous housings, ferrous plates; ferrous brackets.

In any of the embodiments described herein, wherein the detector apparatus is placed at a distance from the exciter coil system wherein a dominant magnetic field detected by the detector apparatus is the eddy current magnetic field.

In any of the embodiments described herein, wherein the exciter coil system and the detector apparatus are positioned at a location that is either (a) within the ferrous structure or (b) exterior to the ferrous structure.

In any of the embodiments described herein, wherein: the exciter coil system is positioned at a location that is either (a) within the ferrous structure or (b) exterior to the ferrous structure, and the detector apparatus is positioned at a location that is either (a) within the ferrous structure or (b) exterior to the ferrous structure.

A method is provided for detecting and quantifying changes in the stress-strain state of ferrous structures. The method includes passing a remote field eddy current probe along the ferrous structure, the probe comprising an exciter coil and a detector coil or multiple detectors spaced from the exciter coil; energizing the exciter coil with a low-frequency alternating current to generate a magnetic field that couples into the ferrous structure to induce eddy currents passing through the ferrous structure, which eddy currents have their own magnetic field that opposes and lags the primary field induced by the exciter coil means; and detecting the magnetic field from the ferrous structure with the detector coil and correlating changes in the detected magnetic field with the stress-strain state of the ferrous structure.

In any of the embodiments described herein, further comprising analyzing the voltage of the detected magnetic field for amplitude and phase lag.

In any of the embodiments described herein, further comprising determining changes in the magnetic permeability of the ferrous structure based on the amplitude and phase lag of the voltage of the detected magnetic field and correlating the changes in magnetic permeability with the level of stress-strain on the ferrous structure.

In any of the embodiments described herein, further comprising energizing the exciter coil with an alternating current in the frequency range of 0.5 to 1000 hertz.

In any of the embodiments described herein, further comprising selecting the ferrous structure from a group including: ferrous pipe, ferrous tubing, ferrous tanks, ferrous pressure vessels, prestressed concrete cylinder pipe, ferrous beams, ferrous housings, ferrous plates, ferrous brackets.

In any of the embodiments described herein, further comprising placing detector coil at a distance from the exciter coil system wherein a dominant magnetic field detected by the detector coil is the eddy current magnetic field.

In any of the embodiments described herein, further comprising positioning the probe structure either (a) within the ferrous structure, or (b) external to the ferrous structure.

In accordance with one embodiment of the of the present invention, a non-transitory computer-readable medium including computer-executable instructions is provided which, when loaded onto a computer, perform a method. The non-transitory computer-readable medium includes computer-executable instructions for: controlling a remote field eddy current probe structure to pass along or through the ferrous structure, the probe comprising an exciter coil and a detector coil spaced from the exciter coil; causing the exciter coil to be energized with a low-frequency alternating current to generate a magnetic field that couples into the ferrous structure to induce eddy currents passing through the ferrous structure, which eddy currents cause the ferrous structure to create its own magnetic field; and controlling the detector coil to detect the magnetic field from the ferrous structure and correlating changes in the detected magnetic field with the stress-strain state of the ferrous structure.

In any of the embodiments described herein, further comprising analyzing the voltage of the detected magnetic field for amplitude and phase lag.

In any of the embodiments described herein, further comprising determining changes in the magnetic permeability of the ferrous structure based on the amplitude and phase lag of the voltage of the detected magnetic field and correlating the changes in magnetic permeability with the level of stress-strain on the ferrous structure.

In any of the embodiments described herein, further comprising positioning the probe structure either (a) within the ferrous structure; or (b) external to the ferrous structure.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 and FIG. 2 show two possible configurations of Exciter and Detector means inside and outside any size of steel (ferrous) pipe or pipeline. These two Exciter-Detector configurations are also suitable for the inspection of PCCP (Prestressed Concrete Cylinder Pipe) shown in FIG. 3.

FIG. 4 shows a dent in a pipe or pipeline wall which represents a local change in the stress-strain state of the pipe without any loss of wall thickness.

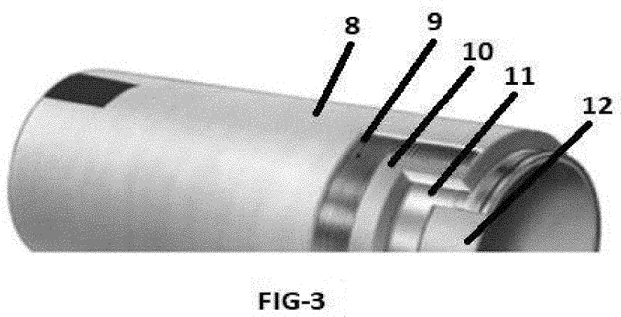

FIG. 5 shows a concrete anchor block commonly used to restrain a pipe underground. An external load, such as soil load, has caused the anchor block to subside, and this causes local areas of stress-strain on the pipe.

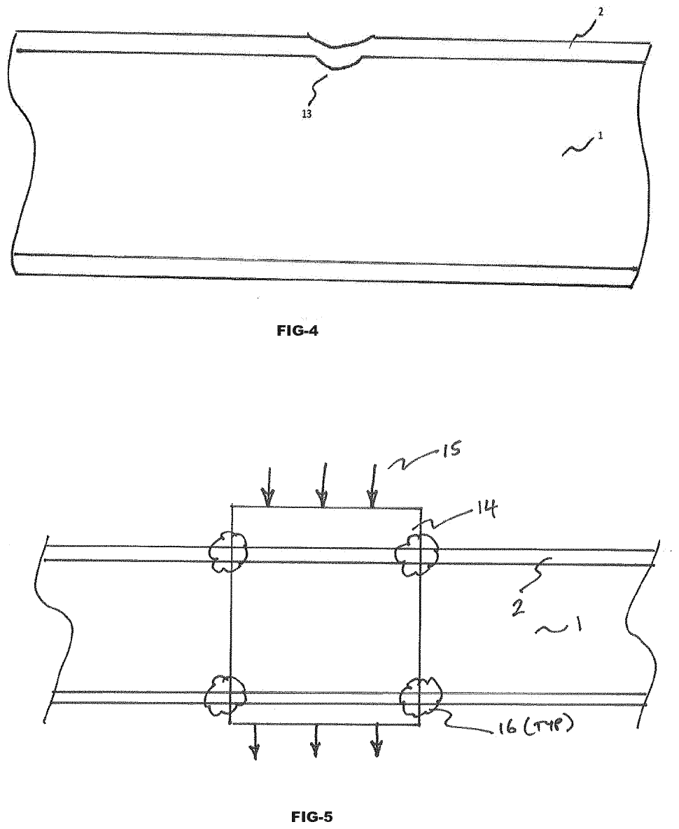

In the PCCP type of pipe shown in FIG. 3, if some of the Pre-Stress wires are cut, the pre-load on the cylinder is locally changed. Again, this is sensed by the instrumentation as a local change in the relative magnetic permeability which indicates the stress-strain state of the pipe.

FIG. 6 shows the fundamental electronics to power the system and measure the stress-strain.

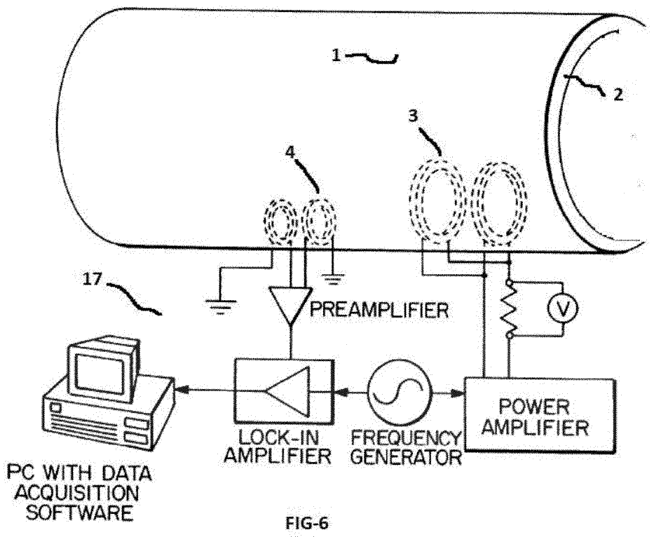

FIG. 7 shows an example of the AC signal applied to the exciter means and how the received signal at the detector means is compared to the exciter means. The variations in time (signal phase) and signal amplitude are shown for nominal pipe and reduced wall thickness pipe.

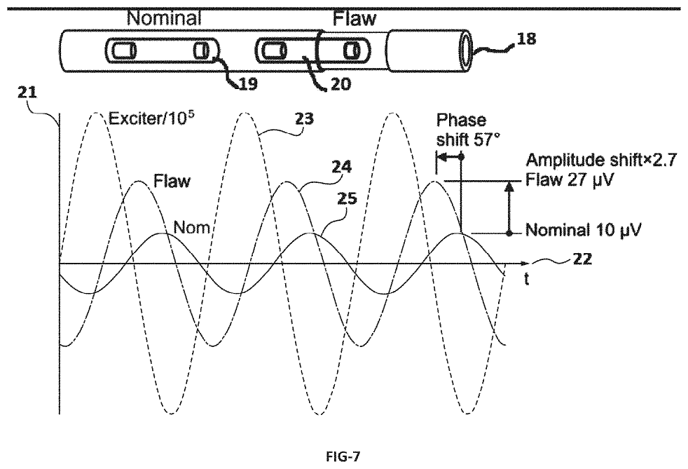

FIG. 8 shows a strip chart and voltage plane representation of the signals from a pipe containing a local change in relative magnetic permeability (stress-strain) and two types of wall loss defects. A RFEC probe pulled through the pipe produced these signals on the instrumentation that is used to gather the data, record it, and display it for analysis.

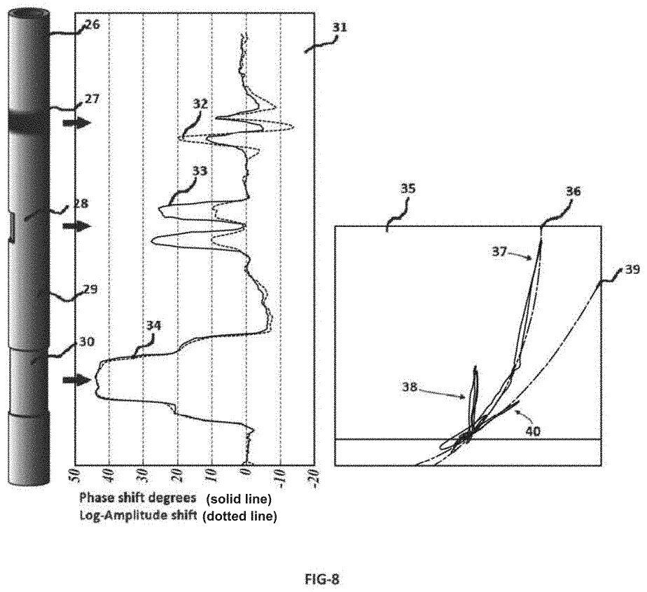

FIG. 9 shows the actual data collected from a pipe at the location of a local stress-strain anomaly (caused by the pipe resting on a rock),

FIG. 10 shows the actual rock 42 and dent 43. The four photographs of the rock and pipe show where the dent was detected by RFEC and confirmed by excavation of the pipeline.

FIG. 11 shows progressive cuts in the pre-stress wires of a C-301E PCCP pipe in an area where the concrete is well bonded. The RFEC data shows no response to the cut wires regardless of how many are cut. This indicates that there was no loss of pre-load on the pipe because the well-bonded concrete takes the load.

FIG. 12 shows progressive cuts in the pre-stress wires of another C-301E PCCP pipe where the concrete is not well bonded. The RFEC data clearly shows the loss of pre-load on the pipe by means of a change in the relative magnetic permeability component of the signal.

### DETAILED DESCRIPTION

The inspection of steel pipelines is well established. In-Line Inspection (ILI) Tools utilize magnetic flux leakage, ultra-sonics, and RFEC to measure the pipe wall for variations in thickness caused by, for example, corrosion or cracks. RFEC is well suited for wall thickness measurements, and is not affected by internal scale, deposits, or liners, and therefore can be used as a non-contact inspection device. The RFEC technique was first patented by McLean in 1951.

Atherton (U.S. Pat. No. 6,127,823), “Electromagnetic Method For Non-Destructive Testing of Prestressed Concrete Cylinder Pipes for Broken Prestressing Wires,” taught that the integrity of pre-stress wires in concrete pressure pipes (PCCP pipes) 8, 9, 10, 11, 12 is critical to the ability of PCCP to maintain its design pressure during operations. If a critical number of pre-stress wires break due to corrosion or embrittlement, the pipe is likely to fail, often catastrophically. Atherton's patent was for a device that could detect broken pre-stress wires from inside the pipe using a technique that he called “Remote Field Eddy Current Transformer Coupling” (RFEC/TC).

The Atherton device induced an electrical current into the helically-wound wires 9 of PCCP pipe by means of an Exciter Coil means 3 placed inside the PCCP pipe in a manner similar to a common transformer. Any breaks in the wires could be detected by a detector coil 4 which was energized primarily by the induced electrical current flowing in the helix of the pre-stress wires and the steel cylinder 11.

Atherton's patent required a separation of the Exciter and Detector coils of at least 2.5 pipe diameters. In this region, the exciter coil's field inside the pipe has reduced to close to zero, but the external field that was induced by the exciter means in the wire helix and the steel cylinder was still strong and was the predominant field. This external field provided energy to the detector coils, again by transformer coupling, which varied depending on the integrity of the wires.

Pure Technologies (U.S. Pat. No. 6,791,318) improved on Atherton's patent by re-orienting the exciter and detector coils to bring them to within one pipe diameter of each other. This coil arrangement was more sensitive to wire breaks and was more efficient because the entire Tool was shorter, lighter, and could travel though a pipe at faster speeds.

In the current disclosure, the detection system does not measure the number of broken wires, but rather it measures the local variations in the amount of stress-strain (“Pre-Load”) that is imputed by the pre-stress wires into the steel cylinder.

Stress-strain in steel components (in this case the steel cylinder in a PCCP pipe) causes the relative magnetic permeability 40, 42 of the pipe to change. During manufacture, the more tension that is applied to the helix wires, the more the steel cylinder is under a compressive load and is therefore able to withstand internal pressure from the liquid or gas that it is transporting. The liquid or gas in the pipeline, in turn, exerts an internal pressure (“hoop stress” load) on the pipe that is the opposite of the compressive load from the pre-stress wires.

The amount of compressive load (Pre-Stress) on the pipe is designed with a safety margin to restrain the hoop stress load that the pipe is designed to withstand in operation. The internal pressure that is found in PCCP pipes comes from the compression of the water or other product that the pipe transports. This may be from the pumps which push the product through the pipes, or the weight of liquid product when the liquid source is at a high elevation. For example, pipes that carry water from mountain reservoirs to coastal treatment plants may vary in elevation by hundreds or thousands of feet. This internal pressure is often known as “head pressure.” Pipeline designers often specify pipes that are near the water source to be significantly thinner than pipes that are near the coast because of the difference in head pressure.

Similarly, pipe designers make pipes of increased thickness 2, 11, wire gauge 9, 44, 48, spacing of the wire helix and thickness of the steel cylinder 11 for pipes that are to withstand higher internal head pressure near the lower elevations on the coast.

When the pre-stress wires break in a PCCP pipe, the thin steel cylinder 11 relaxes and grows in diameter. The degree of relaxation and the growth of the steel cylinder 11 can be minute; however, when enough wires break, the steel cylinder can no longer restrain the internal pressure and the pipe will likely fail. This is an important problem because PCCP pipes are often very large (up to 20′ diameter) and contain vast amounts of water which can cause great damage if released suddenly.

Recently, applicants have shown through experiments performed on actual PCCP pipes removed from service after a critical number of broken wires were detected that the broken wires 45, 48 do not necessarily indicate that the pipe is in immediate danger of failing unless the pipe has also lost its pre-load. What is important to determine is whether the broken wires have allowed the pre-stress load on the cylinder 46, 47 to be reduced. In some cases, the concrete is in such good condition and is so well bonded to the steel cylinder and the pre-stress wires 44 that it retains the preload itself and the pipe is safe to remain in service.

The relaxation of the pre-load on the pipe can be measured by the devices described in this application by measurement of the relative magnetic permeability of the steel cylinder 40, 42. As PCCP pipes are typically very large and therefore expensive to replace, the disclosed system and method offers great value to the pipeline owner because of its ability to identify only those pipes that have reduced pre-load for replacement. PCCP pipe is, however, only one of the applications of the present system and method to detect local changes in the pipe's stress-strain state.

Other examples of local changes in the stress-strain state of pipelines (other than PCCP pipes) are described below, but it may be appreciated that there are many other examples of local changes not discussed herein.

The area of a pipeline that is near a cement anchor block 14 or other pipe restraint when the anchor block has moved due to earthquake, frost heave, or subsidence 15. This places a local load (stress-strain) 16 onto the pipeline. As the load increases the pipeline will deform, causing a dent 13, 43 or wrinkle, and if the load exceeds the tensile strength of the pipe, the pipe will fail.

Side-loads caused by a pipe sliding underground down a side hill. This can happen due to liquefaction of the soil, ground movement, pipe vibration from pumps, and other causes. The result is a local increase in the stress-strain state of the pipe at each end of the slide, where the pipe is restrained.

Point loads caused by the pipe resting on a rock 13, 43. The rock 42 may be supporting the weight of the steel pipeline and the weight of the product inside the pipeline and will impose a local concentration of stress in the pipe wall. Rock damage can lead to dents which often have cracks associated with them.

Bridging can occur when a pipeline is leaking, and the leaking liquid creates a sinkhole under the pipe and the pipe bridges the hole. This results in a change in the local stress of the pipe at each end and at the middle of the bridged section.

External local pressures caused by freezing of the ground around a pipe. When soil freezes, it expands and can squeeze a pipe or exert a side load on the pipe in a local area.

It is these local changes in the stress-strain state of a pipe that affect its relative magnetic permeability property which in turn is detected by the present system and method. The effect of mechanical deformation of a ferrous material on its magnetic permeability is commonly termed the Inverse Magnetostrictive Effect, or the Villari Effect. In materials with a positive saturation magnetostriction, compressive stresses act to increase the relative magnetic permeability of the material, while tensile stresses act to decrease the relative magnetic permeability of the material. In materials with a negative saturation magnetostriction, compressive stresses act to decrease the relative magnetic permeability of the material, while tensile stresses act to increase the relative magnetic permeability of the material. These fluctuations in the relative magnetic permeability quantity are measured and recorded as signal perturbations by the Remote Field Testing technique.

### A. BRIEF DESCRIPTION OF THE INVENTION

According to the invention, an inspection device 3, 4, 17 is provided for ferrous metal objects such as pipes, pipelines, tanks, pressure vessels, and structural components. The device can measure changes in the local stress-strain state of the ferrous metal component from one side of the component, without contact with it, and at distances up to 4″ from the component. One important application is to detect loss of pre-load in pre-stressed concrete cylinder pipes (PCCP) 8, 9, 10, 11, 12. For pipelines such as PCCP, the device may be propelled or pulled through the pipeline to determine stress anomalies and to pinpoint their location along the pipe length.

In one embodiment, the device contains an exciter coil, “exciter means,” 3 which is positioned inside a PCCP pipe. At some distance away (which may vary from a few inches to several pipe diameters) a detector means 4 is positioned to receive the induced field from the exciter means. The detector 4 may be a coil or an array of coils, or it may be any other solid state device that can measure small changes in magnetic fields. The detector means can be a solid-state based sensor like a Hall Effect sensor, a Giant Magneto-Resistive device (“GMR”), or a similar device.

In another embodiment, the exciter means 3 and detector means 4 may be proximal to the outside of a pipe or pipeline or other ferrous component.

In another embodiment, the exciter means 3 may be on one side of a ferrous component and the detector means 4 may be on the other side.

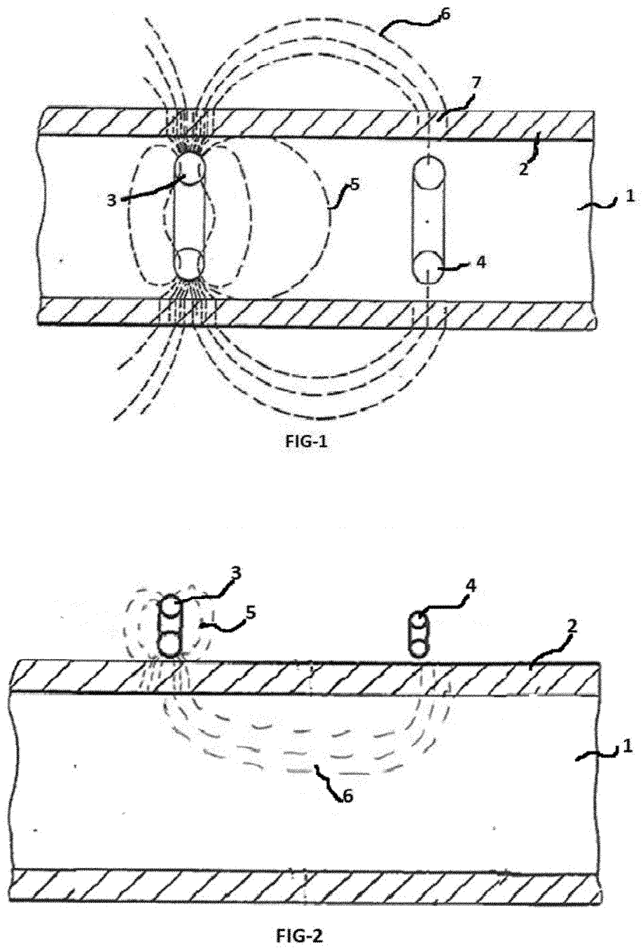

In all embodiments of the invention, the exciter means 3 transmits a low frequency electro-magnetic signal which couples to and is guided by the ferrous component. Much of the electro-magnetic energy is present in the wall of the ferrous component; however, some of the field penetrates through the wall 6. In an RFEC device, the field penetrates the wall twice to travel from the exciter means 3 to the detector means 4 (see FIG. 1). This field is known as a “through transmission” field because it penetrates through the full wall thickness of the ferrous component at least once and usually twice before being received by the detector means 4.

The electromagnetic energy is preferably generated in the frequency range of sub 1 Hz to 1000 Hz. Lower frequencies can penetrate thicker ferrous components and higher frequencies offer increased resolution.

The exciter means 3 may be a coil of copper or aluminum wire that is oriented either co-axially or at an angle to the ferrous structure in order to direct the field in a preferred direction in the pipe wall. The preferred direction may be axial, or circumferential, or radial depending on where it is convenient to place the detector means 4, and what anomalies are to be detected.

In some applications, the change in stress-strain on the pipe is manifested by stress-corrosion cracking (“SCC”) which usually has an axial orientation. In order to detect this type of local stress-strain anomaly, the magnetic component of the field should optimally be at ninety degrees to the crack propagation direction. In this case the exciter means 3 and detector means 4 are ideally oriented to introduce a magnetic field that is normal to the pipe axis.

In a PCCP pipe application, the pre-stress wires 9 are wound onto the cylinder 11 in a helical fashion at high tension to place the steel cylinder into compression and to thereby increase its strength. When the pre-stress wires break, the pre-load on the cylinder is relaxed and the pipe can fail. For this application, the ideal orientation of the exciter-detector array can be co-axial, radial, or circumferential relative to the cylinder. Depending on the make-up of the cylinder (spiral welded, axial welded, riveted etc.), the exciter-detector orientations will vary to place the field along the “magnetic easy access” of the “cylinder.”

In a PCCP pipe, certain circumstances can lead to wire breaks without a loss of preload on the cylinder. This invention can detect a change in the pre-load in PCCP pipe, which is an indication that the pre-stress wires have failed and the pre-load on the steel cylinder has been lost. Conversely, this invention can detect if there has been no loss of pre-load even where the pre-stress wires are broken, for example, due to corrosion or cracking.

There are two common forms of pre-stress wire degradation and failure in PCCP pipe: corrosion failure and cracking failure. In the former case, typically the concrete and/or mortar coating has cracked and has allowed ground water to come into contact with the wires. The wires can then corrode through and release their pre-load on the steel cylinder. Because the concrete has cracked already, it is usually poorly bonded to the wires and cylinder 48 and will typically spall off the outside of the pipe when the wires release their tension. In the latter case, the wires can fail due to hydrogen embrittlement. In this case, the concrete may be in excellent condition: well bonded to the wires and cylinder 44 and cracking failure of the wires does not necessarily release the pre-load on the cylinder and the pipe may be left in service for many years, saving unnecessary costs of replacing pipe that is still fit for service.

### B. EXAMPLES

In order to establish that the RFEC technique can be used to detect and quantify local stress-strain changes, two experiments with prototype equipment were performed:

### Example 1

### PCCP Pipe

Five C-301E PCCP pipes were removed from a pipeline owned by a Texas Water Authority. The pipes had been inspected using the technique patented by Pure Technologies in 2004 (the device that they used contained an exciter means and detector means located within one pipe diameter from each other). Wire breaks had been detected in all five pipes, which exceeded the recommended safety limit for the pipe type and so they were removed from service. Applicant was invited to run its RFEC Tool through the pipes to determine if the wire breaks had resulted in a loss of pre-load on the steel cylinder.

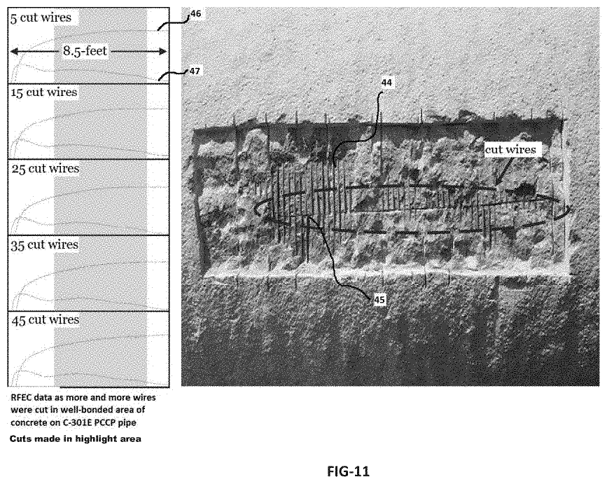

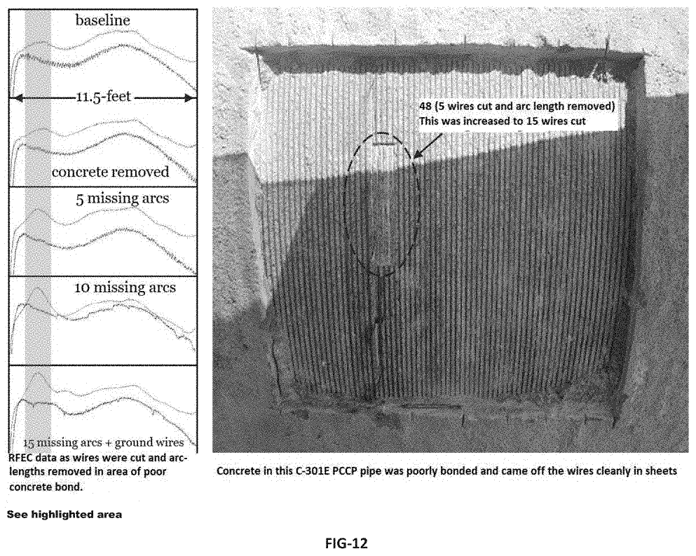

Two areas on different pipes were selected that had no detectable wire breaks. Baseline runs were conducted before and after an area of external concrete was removed to expose the pre-stress wires and to make breaks in them in a controlled manner (FIG. 11 and FIG. 12). The dimensions of the concrete removal area were 12″ and 24″ long×24″ around the pipes. There were no changes in the in-phase or amplitude signals when the RFEC Tool was run past before and after concrete removal on both pipes; however, it was noted that the concrete bonded really well 48 to one of the areas and poorly 44 to the other. The depth of concrete removal was just enough to expose the pre-stress wires.

Wires were cut using a circular saw and after each successive wire cut, the Tool was run past the two areas. After 5 wires were cut into each pipe, the in-phase and amplitude signals in the poorly bonded concrete pipe showed signal changes which increased in size as more wires were cut (FIG. 12). There were no changes in the signals for the well-bonded concrete pipe (FIG. 11).

After ten wires had been cut in both sets of exposed wires, and a length of about 5″ of wire had been removed for each of the ten wires, the wires were electrically reconnected, using short lengths of wire with alligator clips. The RFEC tool was run again and there was no observable change in the signals from either of the two areas from before and after the alligator clips re-connected the wires.

It was also noted that on the pipe that had poorly bonded concrete, when a wire was cut, the wire made an audible ‘snap’ sound and the wire recoiled within the concrete, resulting in a gap of ¼″ to 2″ between the wire ends. On the pipe which had well bonded concrete, there was no “snap” noise when the wires were cut, and the ends of the coil were only the distance apart that was due to the width of the cutting disk (FIG. 11).

Conclusions from the Test on PCCP Pipe

When the concrete has a strong bond to the wires and the steel cylinder it carries or maintains the pre-load on the cylinders by not allowing the wires to recoil and release their pre-load FIG. 11.

When the concrete bond is poor (FIG. 12), the wires are not restrained by the concrete, and they release their pre-load and recoil inside the concrete layer. This releases the pre-load on the cylinder, and this is what is detected by the RFEC tool as a change in relative magnetic permeability.

Wire breaks in areas where the concrete bond is strong, and the pre-load is not released are difficult to detect with the RFEC technique.

The release of pre-load on the thin steel cylinder places the pipe in danger of failure.

The detection and quantification of changes in the local stress-strain state of the cylinder, in this case, a release of compressive force or pre-load, are very viable with the disclosed RFEC technique.

### Example 2

### Oil Pipeline

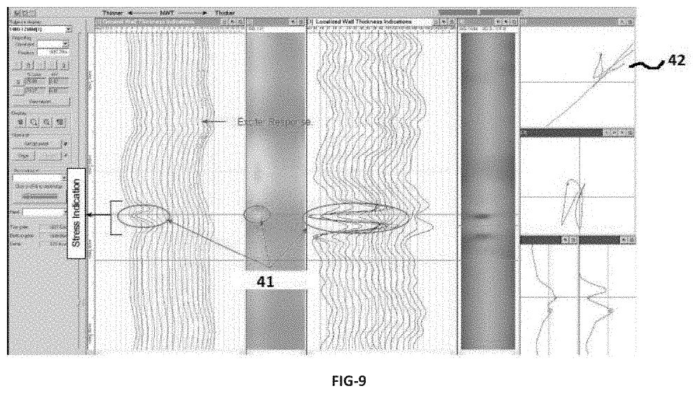

A 6″ diameter steel pipeline was inspected using an RFEC tool and a local magnetic permeability signal was noted (FIG. 9). The signal appeared to be a combination of a local change in stress-strain plus a very deep corrosion spot.

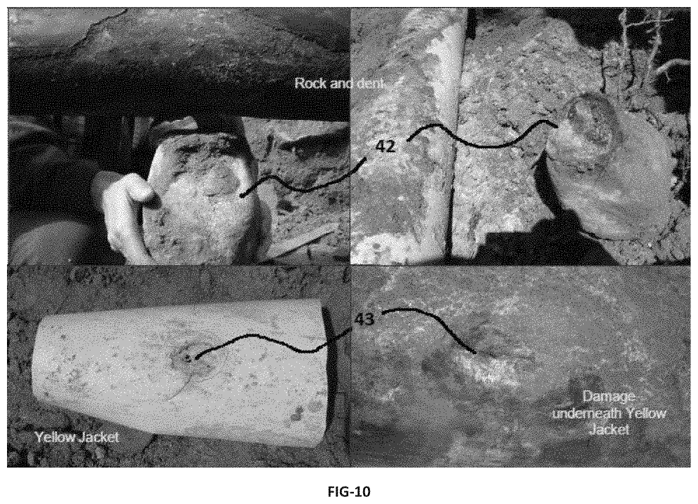

The location of the anomaly was excavated and examined (FIG. 10). It was found that the pipe was resting on a large rock which had dented the pipe. The dent was the source of the local change in stress-strain. This pipeline had been coated on the inside with a hard (but brittle), epoxy liner, and this liner had cracked at the location of the dent, allowing corrosive liquids to attack the inside of the pipe, causing a very deep corrosion pit directly below the cracked epoxy and dent.

Conclusions from the Testing on a Steel Pipeline

An RFEC ILI Tool can detect local areas of change in the stress-strain state of steel pipelines and differentiate the stress-strain signal from the signal that indicates loss of wall thickness.

The signal from a local change of stress-strain state can be analyzed even when a second signal (in this case, from wall loss) is super-imposed on the stress-strain signal (FIG. 9).

Local areas of change in stress-strain can be as important as loss of pipe wall due to corrosion because localized stress-strain can lead to cracking (such as SCC), which in turn can lead to pipeline rupture.

Pipeline rupture is far more dangerous than a corroded through hole which may leak hydrocarbons into the soil. but is unlikely to cause a sudden and massive pipe failure with potential hazards of fire or explosion.

### C. FUNCTIONS OF SYSTEM AND DEVICE

A non-contact measurement system for detecting and quantifying the stress-strain state of ferrous metals and especially pipelines.

A device that detects and quantifies local changes in the relative magnetic permeability of ferrous metal pipes to infer their local stress-strain state.

A device that detects a local change of stress, or a relaxation of stress (“pre-load”) in concrete pressure pipes.

A device that measures the stress-strain state of support beams and columns in structures such as bridges, ships, buildings, storage tanks, pressure vessels and the like.

A measurement technique for assessing the ability of a concrete pressure pipe to contain its design pressure in the presence of a local loss of preload.

An internal device to detect changes in the stress-strain state of a ferrous pipeline.

An external device to detect changes in the stress-strain state of a ferrous pipeline.

A device that works through coatings, fire-proofing, concrete layers, and insulation up to 4″ thick to detect changes in the local stress-strain state of a ferrous metal.

A device that plots the stress-strain state of a pipe or pipeline with respect to its length in order to pinpoint local areas of stress-strain anomalies.

While illustrative embodiments have been illustrated and described, it will be appreciated that various changes can be made therein without departing from the spirit and scope of the invention. For example, the present system and methods can be applied in conjunction with the use of RFEC to detect changes or anomalies in the wall thickness and broken pre-stress wires of the structure being inspected, which as noted above has been the traditional use of RFEC technology. The present disclosure contemplates monitoring not only changes in the structure wall thickness and broken pre-stress wires, but also detecting and quantifying the stress-strain state of the structure simultaneously.

In this regard, applicants note that when ground water comes into contact with the wires and the steel cylinder, the wires will corrode and eventually will break, and the cylinder will either blow out or will corrode through and start leaking. This is what inspection companies that use RFT or NFT have been doing for the last 20+ years. The detection of and realization that permeability signals are embedded within the wall-thickness signals are new to applicants' system and methods as disclosed in this application.

The importance of permeability signals has been realized by applicants because companies have been replacing pipe sections at great expense when there is no corrosion of the wires or cylinder. The wires breaks (typically due to hydrogen embrittlement) have been detected and reported by a RFT or NFT technique; however, even though the wires are broken, applicants have found that the concrete is in good shape and is taking up the load on the cylinder that the wires provided before breaking. The strong concrete, together with the soil load, has resulted in no loss of preload on the cylinder therefore, the pipes can be left in the ground and in service. By monitoring these pipes over time, they can be left in service indefinitely. It is only the lack of change in the relative magnetic permeability signal that can assess whether the preload has been lost.

In order to enhance the permeability signals, multi-frequency techniques are used and the absolute, differential, and signal magnitude (rather than the signal log-amplitude as used in RFT for wall loss detection) are monitored and analyzed.

When true wall loss is encountered, the signal will rotate CCW with increasing frequency; however, permeability signals tend to be very similar in phase angle regardless of frequency.

With respect to monitoring frequency, the frequencies used to detect thickness changes in steel/ferrous pipe (using an RFT Tool from the inside) vary depending on the wall thickness of the steel pipe. For example, for ½″ to ⅝″ steel thickness, exciter frequencies will be in the range of 2 Hz to 10 Hz range. For wall thickness of ¼″ to ⅜″, exciter frequencies will be in the range of 12 Hz to 20 Hz range. For thinner materials, for example, 0.10 inch to 0.20 inch wall thicknesses, the excited frequency may be increased to 20 Hz to 40 Hz. The objective is to set the frequency to that which produces 1 degree of phase angle change for every 1% of wall thickness decrease.

On the other hand, for permeability change monitoring, its only important to have a frequency low enough to penetrate the wall twice and produce a readable signal, so, typically the frequencies used might be 2× those used for wall thickness variation detection. However, permeability analysis can occur at all the same frequencies that are used for RFT, as well as up to two times of the frequencies use for RFT.

An advantage to using a higher frequency for permeability monitoring and analysis is that the inspection speed can be increased.

For external tools or tools that operate in the near field, the “rules” are similar; however, those tools are very sensitive to proximity (lift-off), so it's important for those tools to closely control the lift-off. Internal tools are not as sensitive to lift off, which for internal tools is known as “Fill Factor.”

The embodiments of the invention in which an exclusive property or privilege is claimed are defined as follows:

1. A system for detecting and quantifying changes in the stress-strain state of a ferrous structure, comprising:
an exciter to generate an AC magnetic field that couples into the ferrous structure;
a detector apparatus to detect an eddy current magnetic field resulting from the AC magnetic field generated by the exciter coil system; and
an analyzer that compares the eddy current magnetic field parameters detected by the detector apparatus with the direct AC magnetic field transmitted by the exciter coil system and correlates changes in the parameters of the eddy current magnetic field with the stress-strain on the ferrous structure.

2. A system for detecting and quantifying the condition of a structure that is at least partially composed of ferrous substrate material or ferrous wires for pre-stressing the structure, comprising:
an exciter coil system energized with an alternating current signal to generate an alternating magnetic field that couples into the ferrous structure or ferrous wires;
a detector apparatus to detect an eddy current magnetic field resulting from the alternating magnetic field generated by the exciter coil system; and
an analyzer that compares the eddy current magnetic field parameters detected by the detector apparatus with the alternating magnetic field transmitted by the exciter coil system and correlates changes in the parameters of the eddy current magnetic field with:
(a) changes in the wall thickness of the ferrous structure or breaks in the wire, as well as
(b) changes in the stress-strain on the ferrous structure or the structure that is pre-stressed by the wires.

3. The system of claim 1, wherein the magnetic field parameters that are analyzed include the amplitude and phase lag of the voltage of the eddy current magnetic field detected by the detector apparatus.

4. The system of claim 1, wherein the analyzer:
determines changes in the magnetic permeability of the ferrous structure based on the amplitude and phase lag of the voltage of the eddy current magnetic field detected by the detector apparatus; and
correlates the changes in magnetic permeability with the level of stress-strain on the ferrous structure.

5. The system of claim 1, wherein the frequency of the generated AC magnetic field is in the range of 0.5 to 1000 hertz.

6. The system of claim 1, wherein the ferrous structure is selected from a group including: ferrous pipe, ferrous tubing, ferrous tanks, ferrous pressure vessels, prestressed concrete cylinder pipe, ferrous beams; ferrous housings, ferrous plates; ferrous brackets.

7. The system of claim 1, wherein the detector apparatus is placed at a distance from the exciter coil system wherein a dominant magnetic field detected by the detector apparatus is the eddy current magnetic field.

8. The system of claim 1, wherein the exciter coil system and the detector apparatus are positioned at a location that is either (a) within the ferrous structure or (b) exterior to the ferrous structure.

9. The system of claim 1, wherein: the exciter coil system is positioned at a location that is either (a) within the ferrous structure or (b) exterior to the ferrous structure; and
the detector apparatus is positioned at a location that is either (a) within the ferrous structure or (b) exterior to the ferrous structure.

10. A method of detecting and quantifying changes in the stress-strain state of ferrous structures, comprising:
passing a remote field eddy current probe along the ferrous structure, the probe comprising an exciter coil and a detector coil or multiple detectors spaced from the exciter coil;
energizing the exciter coil with a low-frequency alternating current to generate a magnetic field that couples into the ferrous structure to induce eddy currents passing through the ferrous structure, which eddy currents have their own magnetic field that opposes and lags the primary field induced by the exciter coil means; and
detecting the magnetic field from the ferrous structure with the detector coil and correlating changes in the detected magnetic field with the stress-strain state of the ferrous structure.

11. The method of claim 10, further comprising analyzing the voltage of the detected magnetic field for amplitude and phase lag.

12. The method of claim 10, further comprising determining changes in the magnetic permeability of the ferrous structure based on the amplitude and phase lag of the voltage of the detected magnetic field and correlating the changes in magnetic permeability with the level of stress-strain on the ferrous structure.

13. The method of claim 10, further comprising energizing the exciter coil with an alternating current in the frequency range of 0.5 to 1000 hertz.

14. The method of claim 10, further comprising selecting the ferrous structure from a group including: ferrous pipe, ferrous tubing, ferrous tanks, ferrous pressure vessels, prestressed concrete cylinder pipe, ferrous beams, ferrous housings, ferrous plates, ferrous brackets.

15. The method of claim 10, further comprising placing detector coil at a distance from the exciter coil system wherein a dominant magnetic field detected by the detector coil is the eddy current magnetic field.

16. The method of claim 10, further comprising positioning the probe structure either:
(a) within the ferrous structure; or
(b) external to the ferrous structure.

17. A non-transitory computer-readable medium including computer-executable instructions which, when loaded onto a computer, perform a method, comprising:
controlling a remote field eddy current probe structure to pass along or through the ferrous structure, the probe comprising an exciter coil and a detector coil spaced from the exciter coil;
causing the exciter coil to be energized with a low-frequency alternating current to generate a magnetic field that couples into the ferrous structure to induce eddy currents passing through the ferrous structure, which eddy currents cause the ferrous structure to create its own magnetic field; and
controlling the detector coil to detect the magnetic field from the ferrous structure and correlating changes in the detected magnetic field with the stress-strain state of the ferrous structure.

18. The computer executed method of claim 17, further comprising analyzing the voltage of the detected magnetic field for amplitude and phase lag.

19. The computer method of claim 17, further comprising determining changes in the magnetic permeability of the ferrous structure based on the amplitude and phase lag of the voltage of the detected magnetic field and correlating the changes in magnetic permeability with the level of stress-strain on the ferrous structure.

20. The computer executed method of claim 17, further comprising positioning the probe structure either:
(a) within the ferrous structure; or
(b) external to the ferrous structure.
